(19) United States
(12) Patent Application Publication
(10) Pub. No.: US 2020/0110089 A1
(43) Pub. Date: Apr. 9, 2020

(54) Methods to Identify Leaky Gut Syndrome

(71) Applicant: Maximus Diagnostics Technologies LLC, Little Rock, AR (US)

(72) Inventors: Julia J. Liu, Little Rock, AR (US); Elisabeth M. Davis, Little Rock, AR (US)

(21) Appl. No.: 16/456,131

(22) Filed: Jun. 28, 2019

Related U.S. Application Data
(63) Continuation of application No. PCT/US2017/068628, filed on Dec. 28, 2017.
(60) Provisional application No. 62/440,514, filed on Dec. 30, 2016.

(51) Int. Cl. G01N 33/573; G01N 33/574

(52) U.S. Cl. CPC G01N 33/573; G01N 2800/06; G01N 2333/96466; G01N 33/57419

(57) ABSTRACT

In some embodiments, the invention provides A method for detecting or identifying a leaky gut syndrome in a patient, comprising providing a sample of a GI barrier of the patient; analyzing the sample to determine the status of the GI barrier; and categorizing the patient GI barrier status as normal or abnormal, wherein an abnormal GI barrier status identifies the patient as having leaky gut syndrome. In some embodiments, the patient is human.

### REFERENCE TO RELATED APPLICATION

This patent application is a continuation of International Application No. PCT/US17/068628, filed Dec. 28, 2017, which claims benefit of U.S. provisional application Ser. No. 62/440,514, filed Dec. 30, 2016, the entirety of the contents of each of which are hereby incorporated by reference.

### TECHNICAL FIELD

The present invention relates to fields of biology and medicine.

### BACKGROUND OF THE INVENTION

The gastrointestinal (GI) tract is a series of hollow organs joined in a long, twisting tube from the mouth to the anus of an animal (e.g., a vertebrate animal such as a human). The hollow organs that make up the gastrointestinal (GI) tract are the mouth, the esophagus, the stomach, the small intestine, the large intestine (also known as the colon) which includes the rectum, and the anus. Food (both solid and liquid) enters the mouth and passes to the anus through the hollow organs of the GI tract.

Digestion works by moving food through the GI tract. Food enters the mouth and is broken down with chewing and the digestive juice saliva. It is then swallowed and moves through the esophagus into the stomach where stomach acid further breaks down the food. The digested food then passes into the small intestine, where it mixes with digestive juices, causing large molecules of food to break down into smaller molecules. The body then absorbs these smaller molecules through the walls of the small intestine into the bloodstream, which delivers them to the rest of the body. Waste products of digestion pass through the large intestine and out of the body through the anus as a solid matter called stool or feces.

Bacteria in the GI tract, also called gut flora or microbiome, help with digestion. Parts of the nervous and circulatory systems also play roles in the digestive process. Together, a combination of nerves, hormones, bacteria, blood, and the organs of the digestive system completes the complex task of digesting the food (e.g., solid and liquid) an animal consumes each day.

It is important to note that because the GI tract is a tube, the lumen of the GI tract tube is actually outside of the body of the animal. And, thus, the cells lining the GI tract are actually creating a barrier between the body and the external world. And just as a break or wound in the skin, another barrier between the body and the external world, can be an opening into the body for entering objects (e.g. pathogens such as bacteria or viruses), so too can a break in the GI tract lead to infection.

Leaky gut syndrome (also known as increased intestinal permeability) is caused by breaks in the GI tract.

Often, because leaky gut syndrome starts with a very small break, it is not easily or quickly detected. By the time it is detected, the break in the GI tract may be substantial, and may lead to advanced symptoms in the animal.

Thus, there is a need for improved methods and reagents to rapidly detect leaky gut syndrome.

### SUMMARY OF THE EMBODIMENTS

In a first aspect, the invention provides a method for detecting or identifying a leaky gut syndrome in a patient, comprising: providing a sample of a GI barrier of the patient; analyzing the sample to determine the status of the GI barrier; and categorizing the patient GI barrier status as normal or abnormal, wherein an abnormal GI barrier status identifies the patient as having leaky gut syndrome.

In some embodiments, the patient has or is likely to develop a disease selected from the group consisting of a metabolic syndrome, cancer/neoplasia, an idiopathic inflammatory condition, a neurologic disorder, and a metabolic bone disease. In some embodiments, the patient is human.

In some embodiments, the status of the GI barrier of the patient is analyzed by measuring an amount of activated caspases in intestinal epithelial cells of an intestinal barrier of the patient. In some embodiments, the activated caspase is activated caspase 1, activated caspase 3, or a combination or sum of activated caspase 1 and activated caspase 3. In some embodiments, an increase in the amount of activated caspase by about two to four fold in the patient as compared to the amount of activated caspase in intestinal epithelial cells of an intestinal barrier of one or more healthy volunteers or subjects indicates that the patient GI barrier status is abnormal.

In some embodiments, the activated caspase is a ratio of an amount of expression of activated caspase 1 to an amount of expression of activated caspase 3. In some embodiments, a ratio of activated caspase 1 to activated caspase 3 that is greater than 1.5 to 1 indicates that the patient GI barrier status is abnormal.

In some embodiments, the status of the GI barrier of the patient is analyzed by counting the number of gaps by histological staining of an intestinal surface at the intestinal barrier. In some embodiments, an increase in the number of gaps by about two to four fold in the patient as compared to the number of gaps in an intestinal surface at an intestinal barrier of one or more healthy volunteers indicates that the patient GI barrier status is abnormal.

In some embodiments, the status of the GI barrier is analyzed using confocal laser endomicroscopy or multi-photo confocal microscopy of the GI barrier. In some embodiments, the GI barrier is selected from the group consisting of a buccal mucosa barrier, an oropharyngeal barrier, and an intestinal barrier.

Gastro-intestinal barrier dysfunction or “leaky gut” resulting from microbial imbalances in the gastrointestinal tract are called dysbiosis and may result in the development of disease states such as irritable bowel syndrome and inflammatory bowel disease. Exploration of the complex relationship between our gut microbiome and the intestine have revealed perturbations in the microbial composition and intestinal barrier function may lead to systemic diseases such as metabolic syndromes (1), fatty liver disease (2), obesity (3, 4) neoplasia and cancer including polyps, for example, adenomatous polyps (5), autoimmune or inflammatory conditions (6), neurologic disorders (7), and bone disease (8). The invention provides methods for detecting intestinal barrier function status using tissue samples obtained from patients, either through luminal washing, tissue biopsies, scrapings, brushings, or resection specimens. Samples obtained from a patient with suspected leaky gut related syndromes: metabolic syndromes (including but not limited to diabetes/hypertension/hyperlipidemia), cancer, idiopathic inflammatory conditions (e.g. rheumatoid arthritis), neurologic disorders (e.g. multiple sclerosis) and metabolic bone disease (including but not limited to osteoporosis in adults and primary growth failure in children) can be analyzed using methods provided below for barrier function status.

In another aspect, the invention provides a method for identifying barrier dysfunction using luminal washing/scrapings/brushings using fresh or frozen tissue using a caspase-1 inhibitor (FLICA) as previously reported (Patent # WO 2014/039699 A1).

In another aspect, the invention provides a method of identifying intestinal barrier function status in paraffin-fixed biopsy or resection samples.

In various embodiments of various aspects of the invention, the status of the intestinal barrier is analyzed (or determined) by calculating or measuring an amount of activated caspase 1 expression in intestinal epithelial cells of the intestinal barrier. In some embodiments, the activated caspase is activated caspase 1. In some embodiments, the activated caspase is activated caspase 3. In some embodiments, the activated caspase is a combination of activated caspase 1 and activated caspase 3. In some embodiments, the activated caspase is a ratio of an amount of expression of activated caspase 1 to an amount of expression of activated caspase 3.

In some embodiments, an increase in the amount of activated caspase 1 expression by about two fold in the patient as compared to the amount of activated caspase 1 expression in intestinal epithelial cells of an intestinal sample of healthy volunteers indicates that the patient status is abnormal, or the patient has “leaky gut”. In some embodiments, an increase in the amount of combined activated caspase 1 and 3 expression by between about two to four fold in the patient as compared to the amount of activated caspase expression in intestinal epithelial cells of an intestinal barrier of one or more healthy volunteers indicates that the patient status is indicative of leaky gut for the disease states described above.

In some embodiments, the status of the intestinal barrier is analyzed or determined by counting the number of gaps or extrusion zones in histological staining of an intestinal surface. In some embodiments, an increase in the number of gaps by about two fold in the patient as compared to the number of gaps in an intestinal surface at an intestinal barrier of one or more healthy volunteers indicates that the patient has barrier dysfunction or leaky gut.

In some embodiments of various aspects of the invention, the status of the intestinal barrier is analyzed or determined using confocal laser endomicroscopy, multi-photo confocal microscopy or fluorescent microscopy of the intestinal lining and barrier.

In some embodiments, activated caspase is measured by staining cells of the patient's GI barrier with a detectable marker conjugated to a caspase-1 specific antibody or with a probe comprising a detectable marker conjugated to a caspase-1 inhibitor.

In a second aspect, the invention provides a method for detecting or identifying a leaky gut syndrome in a patient, comprising: staining gastrointestinal (GI) cells of the patient with a detectable marker conjugated to a caspase-1 specific antibody; examining the stained GI cells of the patient for the presence of elevated levels of bound detectable antibody relative to similarly stained GI cells from a healthy individual as evidence of above-normal levels of caspase-1 associated with the patient GI barrier cells, wherein elevated/above-normal levels of caspase-1 in the patient cells as compared to the cells of the healthy subject, identifies the patients as having leaky gut syndrome.

In some embodiments, the caspase-1 specific antibody binds to activated caspase-1.

In some embodiments, the GI barrier is selected from the group consisting of a buccal mucosa barrier, an oropharyngeal barrier, and an intestinal barrier.

In some embodiments, staining comprises the steps of (i) obtaining patient intestinal epithelial cells from the patient by biopsy or aspiration, and (ii) staining the cells in vitro.

In some embodiments, the method further comprises staining the GI barrier cells with a detectable marker conjugated to a caspase-3 specific antibody.

In some embodiments, the antibody binds to activated caspase-3.

In some embodiments, a ratio of activated caspase 1 to activated caspase 3 that is greater than 1.5 to 1 identifies the patient as having leaky gut syndrome.

In some embodiments, an increase in the amount of activated caspase 1 expression by about two fold in the patient as compared to the amount of activated caspase 1 expression in intestinal epithelial cells of an intestinal sample of healthy subjects indicates that the patient status is abnormal, or the patient has “leaky gut”. In some embodiments, an increase in the amount of combined activated caspase 1 and 3 expression by between about two to four fold in the patient as compared to the amount of activated caspase expression in intestinal epithelial cells of an intestinal barrier of one or more healthy subjects identifies the patient has having leaky gut syndrome, as described above.

In some embodiments, the detectable marker is fluorescent, and examining is performed by fluorescence microscopy, multi-photon microscopy, confocal laser endomicroscopy, fluorescence flow cytometry or by using a fluorescence plate reader.

In some embodiments, staining includes applying the detectable marker conjugated to the caspase-1 antibody to intestinal epithelial cells in the patient's intestine, and examining includes visualizing the stained cells endoscopically.

In some embodiments, the detectable marker is a quantum dot.

In some embodiments, the quantum dot has an emission spectra of 625 nm, or in the range of 525 nm to 800 nm or 605 nm and 612 nm.

In some embodiments, the method further comprises the step of identifying dead or dying cells.

In some embodiments, dead or dying cells are identified using the TUNEL assay.

In some embodiments, an increase in the amount of caspase-1 by about two to four fold in the GI barrier cells of the patient as compared to the amount of caspase-1 in GI barrier cells of one or more healthy subjects indicates that the patient has a leaky gut syndrome.

In some embodiments, the patient is human.

In some embodiments, the leaky gut syndrome is neoplasia.

In some embodiments, the leaky gut syndrome is colorectal neoplasia.

In some embodiments, the patient has or is likely to develop a disease selected from the group consisting of a metabolic syndrome, cancer/neoplasia, an idiopathic inflammatory condition, a neurologic disorder, and a metabolic bone disease.

In some embodiments, the method detects activated caspase-1 and/or activated caspase-3.

In some embodiments, the method uses paraffin fixed biopsy or resection samples.

In a third aspect, the invention provides a method for detecting or identifying a leaky gut syndrome in a patient, comprising: staining gastrointestinal (GI) cells of the patient with a probe comprising detectable marker conjugated to a caspase-1 inhibitor; examining the stained GI cells of the patient for the presence of elevated levels of bound detectable probe relative to similarly stained GI cells from a healthy individual as evidence of above-normal levels of caspase-1 associated with the patient GI barrier cells, wherein elevated levels of caspase-1 identifies the patients as having leaky gut syndrome.

In some embodiments, the probe is a conjugate of a caspase-1 inhibitor and a fluorochrome.

In some embodiments, the caspase-1 inhibitor is FLICA.

In some embodiments, the probe is a conjugate of the tetrapeptide YVAD (SEQ ID NO: 1) and a fluorochrome.

In some embodiments, the probe comprises Ac-YVAD (tyr-val-ala-asp)-CMK (SEQ ID NO: 1).

In some embodiments, the probe has the structure Alexa Fluor 488-GGGG-YVAD-FMK (SEQ ID NO: 2).

### BRIEF DESCRIPTION OF THE DRAWINGS

The file of this patent contains at least one drawing executed in color. Copies of this patent with color drawings will be provided by the Office upon request and payment of the necessary fee.

FIG. 1 is a schematic diagram showing pyroptotic extrusion of epithelial cells mediated by caspase-1 activation from the intestinal lining. Also shown are epithelial gaps or extrusion zones between the cells left after cells are extruded. The gaps are not sealed and remain open to the gut lumen, thereby resulting in leaky gut.

FIG. 2 is a photographic image showing staining for activated caspase 1 in intestinal epithelial cells (IECs). The white arrow heads points to IECs staining positive for activated caspase 1, and the red arrowhead points to intra-epithelial lymphocytes staining positive for CD3 (a T-cell marker).

FIGS. 3A and 3B are photographic images showing the staining of intestinal epithelial cells (IECs) for nuclear fragmentation using a commercially available TUNEL stain (FIG. 3A), which will stain both activated caspase 1 and activated caspase 3 (FIG. 3B). In FIG. 3A, the white arrows point to TUNEL-positive cells (i.e., cells with nuclear fragmentation). In FIG. 3B, the white arrows point to activated caspase-3 positive cells.

FIG. 4 is a graph showing the percentage of activated Caspase-1 positive epithelial cells obtained by mucosal biopsy from healthy subjects versus patients with leaky gut and colorectal neoplasia.

FIGS. 5A-5C present representative images of intestinal biopsy samples from a healthy patient (5A), a patient with colorectal cancer (5B) stained using primary monoclonal activated caspase-1 antibody; and a patient with leaky gut and colorectal neoplasia stained using the quantum dot conjugated antibody (5C). White arrowheads indicate caspase-1 positive intestinal epithelial cells in the mucosal biopsy samples.

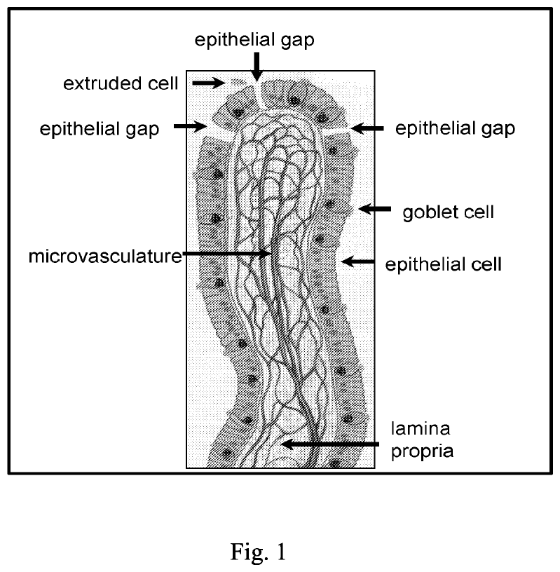

### DETAILED DESCRIPTION OF SPECIFIC EMBODIMENTS

The invention stems, in part, from the discovery that leaky gut syndrome can be identified by analyzing samples obtained from the gastro-intestinal epithelium (esophageal, gastric, and intestinal, including rectum), oropharynx, or buccal mucosa of a patient.

The published patents, patent applications, websites, company names, and scientific literature referred to herein establish the knowledge that is available to those with skill in the art and are hereby incorporated by reference in their entirety to the same extent as if each was specifically and individually indicated to be incorporated by reference. Any conflict between any reference cited herein and the specific teachings of this specification shall be resolved in favor of the latter.

Terms defined or used in the description and the claims shall have the meanings indicated, unless context otherwise requires. Technical and scientific terms used herein have the meaning commonly understood by one of skill in the art to which the present invention pertains, unless otherwise defined. Any conflict between an art-understood definition of a word or phrase and a definition of the word or phrase as specifically taught in this specification shall be resolved in favor of the latter. As used herein, the following terms have the meanings indicated. As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As discussed above, the gastrointestinal (GI) tract is a hollow tube that serves as a barrier between the body and the outside world environment that exists in the lumen of the tube. In the lumen of the tube, which starts with the mouth and ends with the anus, food (including solid and liquid food) is digested, nutrients extracted through the intestinal barrier, and feces are formed to be ejected through the anus.

The initial surface between the outside world and the body is the buccal mucosa barrier, which is the inside lining of the cheeks and lips. There is a mucosal lined surface at the back of the throat as well that serves as a barrier. This is referred to as an oropharyngeal barrier.

The gastrointestinal (GI) barrier is a single-cell layer of epithelial cells that constitutes the largest and most important barrier against the external environment. The gastrointestinal barrier acts as a selectively permeable barrier, permitting the absorption of nutrients, electrolytes, and water while maintaining an effective defense against intraluminal toxins, antigens, and enteric flora. The lining of the gastro-intestinal tract which makes up the epithelial cells undergoes continuous physiologic renewal: stem cells located at the base of the crypts mature and migrate up the epithelial surface. The mature epithelial cells are eventually shed at the tip of the surface or villi in the intestine. Note that when the epithelial cells of the GI barrier are being analyzed, only the epithelial cells at the surface of the GI barrier are analyzed for epithelial gaps and expression of activated caspase 1 and/or caspase 3, not the cells at the crypts.

Each of the buccal mucosa barrier, the oropharyngeal barrier, and the intestinal barrier is referred to as a “GI barrier”, and collectively as “GI barriers”.

Accordingly, in a first aspect, the invention provides a method for detecting a leaky gut syndrome in a patient, comprising: providing a sample of a GI barrier of the patient; analyzing the sample to determine the status of the GI barrier; and categorizing the patient GI barrier status as normal or abnormal, wherein an abnormal GI barrier status identifies the patient as having leaky gut syndrome.

As used herein, by “patient” is simply meant any patient or subject from whom a GI barrier sample is taken. In some embodiments, the patient may have no symptoms whatsoever and may have given a sample during a routine wellness check, yearly physical, or routine colonoscopy. In some embodiments, the patient may have some symptoms related to bowel disorder, such as symptoms for irritable bowel syndrome or inflammatory bowel disease. The most common types of inflammatory bowel disease (IBD) are ulcerative colitis, Crohn's disease and indeterminate colitis. A subset of recently described IBD-like inflammatory colitis is chemotherapy-induced colitis and is also included herein as a type of inflammatory bowel disease.

By “sample” simply means any sample containing cells from the patient. For example, for a buccal mucosa barrier sample or an oropharyngeal barrier sample, scrapings from the inner cheek or the back of the throat, respectively, can be used as samples. For a sample from an intestinal barrier, any biopsy tissues taken during a colonoscopy or an endoscopy can be used as samples. Samples also include luminal washing/scrapings/brushings using fresh or frozen tissue sample using a caspase-1 inhibitor (FLICA) as previously reported (See PCT Patent Publication No. WO 2014/039699, the entirety of which is incorporated herein by reference).

As used herein, by “leaky gut syndrome” or simply “leaky gut” is meant a condition in which a break occurs in the GI barrier, thereby exposing the inside of the body to the external environment present in the lumen of the GI tract. Leaky gut syndrome is also known as increased intestinal permeability, and as a result of breaks in the GI tract, objects that are not supposed to be absorbed through the GI tract and into the body are, in fact, allowed entry into the body. These foreign objects can be a single molecule, such as an incompletely digested food molecule, or can be as large as a pathogen, such as a bacteria or virus.

Note that in leaky gut syndrome, the break can be very small (e.g., to only allow larger molecules such as simple sugars to enter the body from the lumen of the GI tract), or can be larger (e.g., to allow bacteria and cells to enter the body from the lumen of the GI tract). Ideally, leaky gut syndrome will be detected while the break is still very small, and before the break is large enough to allow entry of a large foreign body, such as a virus or bacteria.

Leaky gut syndrome can be caused, for example, by increased intestinal permeability or intestinal hyperpermeability.

Patients with leaky gut syndrome may already have or may develop symptoms of irritable bowel syndrome, inflammatory bowel disease (e.g., Crohn's disease, ulcerates colitis, indeterminate colitis and chemotherapy-induced colitis), celiac disease, allergy (e.g., food allergy), asthma, autism, chronic fatigue syndrome, lupus, metabolic syndromes (including, but not limited to, diabetes, hypertension, and hyperlipidemia), neoplasia or cancer, idiopathic inflammatory conditions (e.g. rheumatoid arthritis), neurologic disorders (e.g. multiple sclerosis), migraines, psoriatic arthritic, autoimmune diseases such as rheumatoid arthritis and psoriasis, metabolic bone disease (including but not limited to osteoporosis in adults and primary growth failure in children), and other disease indications that systemic or related to the GI tract. Leaky gut can also arise in patients, such as elderly patients or children, compromising the ability of the patient to absorb nutrients from consumed food.

In some embodiments, leaky gut results when tight junctions between individual cells at the GI barrier become loosened, allowing particles and potentially microbes to pass through the junction from the lumen and into the body. The loosening of the tight junctions may be due, for example, crenation or shrinkage of the cells, thereby widening the junction between the crenated cell and its adjacent cell.

In some embodiments, the leaky gut results from other types of damage to the cells at the GI barrier. For example, the cells at the barrier may become inflamed or may start expressing proteins involved in programmed cell death (e.g., apoptosis, pyroptosis and necroptosis).

In some embodiments, the status of the GI barrier of a patient sample can be analyzed by measuring or calculating the amount of activated caspase expressed in epithelial cells at the intestinal surface of the GI barrier. For example, the amount of activated caspase can be determined by staining a sample (e.g., a biopsy sample) from the patient with a detectably labeled antibody that specifically binds to an activated caspase molecule (e.g., activated caspase 1 or activated caspase 3). The amount of activated caspase can also be determined by staining a sample from the patient with a detectably labeled peptide that binds to activated caspase. It should be noted that by being detectably labeled, the peptide or antibody can be directly labeled (e.g., with a fluorescent label or chromatogenic tag) or can be detected by being bound during secondary staining with an detectably labeled secondary antibody (e.g., the anti-caspase antibody is a murine monoclonal antibody and the secondary antibody is a fluorescently labeled rabbit anti-mouse antibody).

In some embodiments, the activated caspase is activated caspase 1. In some embodiments, the activated caspase is activated caspase 3. In some embodiments, the activated caspase is a combination of activated caspase 1 and activated caspase 3. In some embodiments, the activated caspase is a ratio of an amount of expression of activated caspase to an amount of expression of activated caspase 3. For example, in a normal healthy volunteer, the ratio of activated caspase 1 to activated caspase 3 is 1 to 1. However, in a patient with an abnormal GI barrier, the ratio of activated caspase 1 to activated caspase 3 is 1.5 to 1, or greater than 1.5 to 1. That is, the expression of activated caspase 1 is greater than or equal to 1.5 fold higher than the expression of activated caspase 3 in a patient with an abnormal GI barrier.

Typically, epithelial cells at the intestinal barrier of people who do not have inflammatory bowel disease (e.g., do not have IBD symptoms) express certain level of activated caspases (e.g., express activated caspase 1 or activated caspase 3 at between 0.5 to 1%). Such people who do not have gastrointestinal symptoms may be referred to as a healthy volunteer. Accordingly, in some embodiments, an amount of activated caspase expression in a patient that is more than two to four fold higher than the amount of activated caspase expression in epithelial cells of a GI barrier of one or more healthy volunteers indicates that the patient GI barrier status is abnormal and that the patient has leaky gut.

Note that the amount of activated caspase expressed by a healthy volunteer will depend upon several factors including the reagent used to detect the activated caspase (e.g., the peptide inhibitor, Ac-YVAD (tyr-val-ala-asp)-CMK (SEQ ID NO: 1), from Enzo described below that inhibits activated caspase 1 or an antibody that specifically binds to activated caspase 1 such as the antibody from Cell Signaling Technology, Inc. described below).

In some embodiments, the amount of activated caspase expression in epithelial cells of a GI barrier of a healthy volunteer is 1%. Thus, if a patient has 2% activated caspase 1 expression (i.e., has 2 out of 100 epithelial cells expressing activated caspase 1), that patient will be categorized as having an abnormal GI status and therefore as having leaky gut because the patient has a 2 to 4 higher expression of activated caspase 1 than the healthy volunteer.

In some embodiments, the amount of activated caspase expression in intestinal epithelial cells of a GI barrier of a healthy volunteer is approximately 0.5%. Thus, if a patient has more than 1% activated caspase 1 expression (i.e., has 1 out of 100 epithelial cells expressing activated caspase 1), that patient will be categorized as having an abnormal GI status and therefore as having leaky gut because the patient has a 2 to 4 fold higher expression of activated caspase 1 than the healthy volunteer.

Where there is no number or percentage value available for “the amount of activated caspase expression in epithelial cells of a GI barrier of one or more healthy volunteers”, that amount shall understood to be in the range of about 0.5 to 1.0 cells out of 100 or 0.5% to 1.0% expression for caspase-1 and caspase-3 individually, and 1 to 2% expression for total caspase positive cells.

Where there is no number or percentage value available for “the amount of activated caspase 1 expression in epithelial cells of a GI barrier of one or more healthy volunteers”, that amount shall understood to be in the range of about 0.5 cells out of 100 or 0.5% expression for activated caspase-1 (or 0.005).

In some embodiments, the status of the GI barrier of a patient sample can be analyzed by measuring or calculating the number of gaps in routine histological staining of the intestinal lining. For example, the residual spaces left in between cells in the intestinal surface after extrusion of epithelial cells, also called extrusion zones, can be counted on well preserved intestinal specimens and normalized to the total number of epithelial cells to reflect the barrier status. The samples can be stained using conventional histologic staining techniques, including but not limited to hematoxylin and eosin stain, alcian blue and nuclear fast red.

In some embodiments, the status of the GI barrier is determined by measuring gap density (i.e., number of gaps) using confocal endomicroscopy of the GI surface. For example, the patient samples (e.g., intestinal samples collected during endoscopy) can be stained with a nuclear (such as DAPI) stain and cytoskeletal (e.g., actin) stain and imaged using multi-photon confocal microscopy ex-vivo.

Ordinarily, a healthy volunteer will have very limited GI barrier gaps, and so that number of gaps in a healthy volunteer is ordinarily under 1 gap per 100 intestinal epithelial cells.

However, where there is no number or percentage value available for the number of gaps or the gap density of one or more healthy volunteers, that amount shall understood to be approximately 1 gap per 100 intestinal cells, or approximately 1%.

In some embodiments, the mucosal (or epithelial) barrier status or the degree of GI barrier dysfunction can be characterized by a combination stain for activated caspase-1 and/or activated caspase-3 of intestinal epithelial cells, and anti-CD3 of intraepithelial lymphocytes. The total number of intestinal epithelial cells can be quantitated using nuclear stains (e.g., DAPI). The staining methods are detailed in Example 2 “Staining protocol for paraffin-embedded mucosal biopsy samples” below.

The degree of GI barrier dysfunction can be derived by either the total number of activated caspase-1 positive cells normalized to the total number of intestinal epithelial cells (e.g., as determined by nuclear stain); or a relative ratio of activated caspase-1 positive to activated caspase-3 positive cells, or a combination of activated caspase-1 positive and activated caspase-3 positive cells normalized to the total number of intestinal epithelial cells.

The GI barrier status or the degree of barrier dysfunction can also be characterized by a combination stain including a TUNEL stain which will stain positive for both activated caspase-1 and activated caspase-3 epithelial cells, minus the activated caspase-3 positively stained cells; with or without anti-CD3 stain to differentiate intraepithelial lymphocytes from intestinal epithelial cells. The total number of intestinal epithelial cells can be quantitated using nuclear stains, e.g. DAPI. The staining methods are detailed in Example 3 “TUNEL staining protocol for paraffin-embedded mucosal biopsy samples using commercially-available staining kits”, below.

In some embodiments, the GI (e.g., epithelial or mucosal) barrier dysfunction can alternatively be characterized by staining for active interleukin 1-beta (IL-1β) and/or IL-18, both of which are surrogate markers of activated caspase-1. Antibodies that specifically bind to active (i.e., mature) interleukin 1-beta (IL-1β) and antibodies that specifically bind to IL-18 are known (see, e.g., Cleaved-IL-1β (Asp116) (D3A3Z) Rabbit mAb #83186, Cell Signaling Technology, Inc., Danvers, Mass., USA, and anti-IL18 antibody (ab71495), Abcam, Cambridge, Mass., USA).

In vivo, the GI surface may be stained with intravenous dye (e.g., fluorescein) with or without a nuclear stain (e.g., acriflavine), and imaged using confocal laser endomicroscope. Gap density on confocal laser endomicroscopy is a validated measure of extrusion zones.

The status of the GI barrier is significantly compromised in inflammatory bowel disease (IBD) patients as compared to the status of an intestinal barrier from a healthy volunteer (e.g., a person, aged 18 to 70) who does not have gastrointestinal symptoms.

The invention also provides a method for detecting or identifying a leaky gut syndrome in a patient, comprising: staining gastrointestinal (GI) cells of the patient with a detectable marker conjugated to a caspase-1 specific antibody; examining the stained GI cells of the patient for the presence of elevated levels of bound detectable antibody relative to similarly stained GI cells from a healthy individual as evidence of above-normal levels of caspase-1 associated with the patient GI barrier cells, wherein elevated/above-normal levels of caspase-1 in the patient cells as compared to the cells of the healthy subject, identifies the patients as having leaky gut syndrome. The caspase-1 specific antibody recognizes activated caspase-1.

In some embodiments, the GI barrier can be any one of a buccal mucosa barrier, an oropharyngeal barrier, and an intestinal barrier.

In some embodiments, staining includes the steps of (i) obtaining patient intestinal epithelial cells from the patient by biopsy or aspiration, and (ii) staining the cells in vitro. In some embodiments, the method further comprises staining the GI barrier cells with a detectable marker conjugated to a caspase-3 specific antibody wherein, the antibody detects activated caspase-3.

In a normal healthy subject, the ratio of activated caspase 1 to activated caspase 3 is 1 to 1. However, in a patient with an abnormal GI barrier and a leaky gut syndrome, the ratio of activated caspase 1 to activated caspase 3 is 1.5 to 1, or greater than 1.5 to 1. That is, the expression of activated caspase 1 is greater than or equal to 1.5 fold higher than the expression of activated caspase 3 in a patient with an abnormal GI barrier or a leaky gut syndrome.

In some embodiments, an increase in the amount of activated caspase 1 expression by about two fold in the patient as compared to the amount of activated caspase 1 expression in intestinal epithelial cells of an intestinal sample of healthy subjects indicates that the patient status is abnormal, or the patient has “leaky gut”. In some embodiments, an increase in the amount of combined activated caspase 1 and 3 expression by between about two to four fold in the patient as compared to the amount of activated caspase 1 and 3 expression in intestinal epithelial cells of an intestinal barrier of one or more healthy subjects identifies the patient has having leaky gut syndrome, as described above.

In some embodiments, the detectable marker is fluorescent, and examining is performed by fluorescence microscopy, multi-photon microscopy, confocal laser endomicroscopy, fluorescence flow cytometry or by using a fluorescence plate reader.

In some embodiments, staining includes applying the detectable marker conjugated to the caspase-1 antibody to intestinal epithelial cells in the patient's intestine, and examining includes visualizing the stained cells endoscopically. In some embodiments, the detectable marker is a quantum dot, for example, having an emission spectra of 625 nm, or in the range of 525 nm to 800 nm or 605 nm and 612 nm.

In some embodiments, the method further comprises the step of identifying dead or dying cells, for example, using the TUNEL assay. TUNEL (Terminal deoxynucleotidyl transferase dUTP nick end labeling) staining is a method for detecting DNA fragmentation by labeling the terminal ends of nucleic acids. Since apoptosis causes fragmentation of DNA, the TUNEL assay is a common method for DNA fragmentation that results from apoptotic signaling cascades. The assay relies on the presence of nicks in the DNA which can be identified by terminal deoxynucleotidyl transferase or TdT, an enzyme that will catalyze the addition of dUTPs that are secondarily labeled with a marker.

In some embodiments, an increase in the amount of caspase-1 by about two to four fold in the GI barrier cells of the patient as compared to the amount of caspase-1 in GI barrier cells of one or more healthy subjects indicates that the patient has a leaky gut syndrome.

In some embodiments, the patient is a mammal, for example a human.

In some embodiments, the leaky gut syndrome is neoplasia, for example, colorectal neoplasia. In some embodiments, the patient has or is likely to develop a disease selected from the group consisting of a metabolic syndrome, cancer, neoplasia, an idiopathic inflammatory condition, a neurologic disorder, and a metabolic bone disease.

In some embodiments, the method detects activated caspase-1 and activated caspase-3.

In some embodiments, the method uses paraffin fixed biopsy or resection samples.

In a third aspect, the invention provides a method for detecting or identifying a leaky gut syndrome in a patient, comprising: staining gastrointestinal (GI) cells of the patient with a probe comprising detectable marker conjugated to a caspase-1 inhibitor; examining the stained GI cells of the patient for the presence of elevated levels of bound detectable probe relative to similarly stained GI cells from a healthy individual as evidence of above-normal levels of caspase-1 associated with the patient GI barrier cells, wherein elevated levels of caspase-1 identifies the patients as having leaky gut syndrome. In some embodiments, the probe is a conjugate of a caspase-1 inhibitor and a fluorochrome. In some embodiments, the caspase-1 inhibitor is FLICA. In some embodiments, the probe is a conjugate of the tetrapeptide YVAD and a fluorochrome. In some embodiments, the probe comprises Ac-YVAD (tyr-val-ala-asp)-CMK. In some embodiments, the probe has the structure Alexa Fluor 488-GGGG-YVAD-FMK.

The following examples are not meant to limit the invention in any way.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1

### Preparation of Paraffin-Embedded Mucosal Biopsy Samples

Paraffin embedded human tissue blocks were sectioned at 5 μm and the tissue sections were mounted onto glass slides.

### Example 2

### Staining Protocol for Paraffin-Embedded Mucosal Biopsy Samples

Described below is a three step protocol for staining paraffin-embedded mucosal biopsy samples.

### Step I. Deparaffinization

Samples were obtained and mounted on slides as described in Example 1.

The slides were placed in a rack, and the following sequential washes were performed in Coplin jars or other suitable containers:
Wash 1. Xylene: 2×5 minutes
Wash 2 and 3. 100% ethanol: 2×5 minutes
Wash 4. 95% ethanol: 3 minutes
Wash 5. 70% ethanol: 3 minutes
Wash 6. 50% ethanol: 3 minutes
Wash 7. Distilled H₂0: 2×3 minutes

The slides were kept in the distilled water until ready to perform antigen retrieval. In some embodiments, the slides were not allowed to dry from this point onwards, as drying out could cause non-specific antibody binding and therefore high background staining on the tissue.

### Step II. Antigen Retrieval

1. A water bath and antigen retrieval solution (10 mM sodium citrate buffer) were pre-heated to 95° C. The 10 mM sodium citrate buffer was 10mM sodium citrate, 0.05% Tween 20, pH 6.0, and was made as follows: Tri-sodium citrate (dihydrate) 2.94 g was combined with 1000 ml distilled water and mixed to dissolve. The pH was adjusted to 6.0 with 1N HCl. 0.5 ml Tween 20 was added to the solution, and the solution was mixed well, and stored at 4° C.

2. The slides were placed in pre-heated antigen retrieval solution in a container (enough to cover the slides by about 1 to about 8 centimeters). As glass containers may crack in the heat, glass containers are not preferable. In some embodiments, a plastic tupperware container or other type of plastic container with a lid to prevent evaporation was used. In some embodiments, an empty box with a lid (e.g., a box that is used to hold pipet tips for a micropipetter) was used. In some embodiments, a weight was added to the cover of the container to prevent the container from floating/moving around in the antigen retrieval solution.

3. The slides were incubated for 20 minutes at 95° C.

4. When 20 minutes had elapsed, the container and slides were removed from the water bath. The slides were allowed to cool at room temperature, still immersed in the antigen retrieval solution, before removing them from the container.

The immunohistochemical staining protocol (i.e., Step III) described below was then performed.

### Step III. Immunostaining

All incubations were carried out in a humidified chamber to avoid drying of the tissue.

A shallow, plastic box with a sealed lid and wet tissue paper in the bottom was used for immunostaining. The slides were kept off the paper and laid flat so that the reagents did not drain off.

1. The slides were washed in 1× PBS (phosphate buffered saline) with 0.025% Triton X-100 for 5 minutes with gentle agitation. A second wash was performed for a total of 2 washes.

2. The slides were removed from the wash buffer and the excess liquid from the slides was dried using a Kimwipe or other delicate task wipe with low lint and low electrostatic discharge. A PAP pen or other similar pen was used to draw a circle around the tissue to create a hydrophobic barrier around the sample.

3. Approximately 100 μL of blocking solution was pipetted onto the tissue, ensuring that the tissue section was completely covered, and the tissue was incubated at room temperature for 2 hours. The blocking solution contained: 1× PBS with 10% normal goat serum and 1% BSA (bovine serum albumin).

4. A Kimwipe was used to blot off any excess blocking solution from the tissue and approximately 100 μL of primary antibody solution was added to each slide. The slides were incubated overnight at 4° C. The primary antibody solution contained 1× PBS with 1% BSA and Caspase-1 p20 antibody at a 1:250 dilution (for example, the Cleaved caspase-1 (Asp297)(D57A2) rabbit mAb available from Cell Signaling Technologies (Danvers, Mass.), cat# 4199), and with CD3e antibody at a 1:100 dilution (using, for example, the CD3e/CD3 epsilon human antibody (SPV-T3b), raised in mouse; Invitrogen (Carlsbad, Calif.) cat# 07-0303).

In certain embodiments, staining of activated caspase 1 was accomplished by immunoblotting with a peptide inhibitor, such as the Ac-YVAD (tyr-val-ala-asp)-CMK (SEQ ID NO: 1) inhibitor commercially available from Enzo Life Sciences, Farmingdale, N.Y., and described in PCT Publication No. WO2014/039699 and US patent publication no. 2015/0202329, both incorporated by reference herein in their entireties. The peptide Ac-YVAD-CMK (Ac-Tyr-Val-Ala-Asp-chloromethylketone) (SEQ ID NO: 1) is a cell permeable, irreversible inhibitor of caspase-1.

5. Excess primary antibody solution was drained from the slides and the slides were washed in 1× PBS for 5 minutes with gentle agitation. This step was repeated twice for a total of 3 washes.

6. Approximately 100 μL of secondary antibody solution was pipetted onto the tissues and the tissues were incubated at room temperature (e.g., 25° C.) for 1 hour in the dark. The secondary antibody solution contained 1× PBS with 1% BSA and Goat anti-rabbit AlexaFluor 488 at 1:3000 dilution (using, for example, the Goat anti-rabbit (H+L) Superclonal secondary antibody, AlexaFluor conjugate 488; Invitrogen, cat# PIA27034), and with Goat anti-mouse AlexaFluor 555 at a 1:3000 dilution (using, for example, the Goat anti-mouse IgG (H+L), AlexaFluor conjugate 555; Invitrogen, cat# A21424).

7. Excess antibody solution was blotted from the slides, and the slides were washed in 1× PBS for 5 minutes, with gentle agitation. This step was repeated once for a total of two washes. The washes were performed in the dark.

8. Slides were incubated in 1× PBS containing 0.3 μg/mL (0.654 nM) DAPI(4′,6-Diamidino-2-Phenylindole, Dilactate), commercially available for example, from Molecular Probes, cat# D3571 for 10 minutes with gentle agitation in the dark. For example, 12 μL of 5 mg/mL DAPI stock in 200 mL PBS was used for a final wash and stain in one step.

9. Excess liquid was drained, tissue paper was used to wipe around the sections, and the coverslips were mounted on the tissue. This was done by using a mounting agent such as, for example, ProLong Diamond Antifade Mountant (Molecular Probes (Eugene, Oreg.), cat# P36970). The slides were allowed to cure (i.e., the tissue with the mounting agent set and hardened with time) overnight at room temperature in the dark before imaging.

Slides were imaged using either multi-photon microscopy or fluorescent microscopy, using wavelengths corresponding to the fluorochrome used. In certain embodiments, confocal laser endomicroscopy was performed on patient samples during the time of endoscopy.

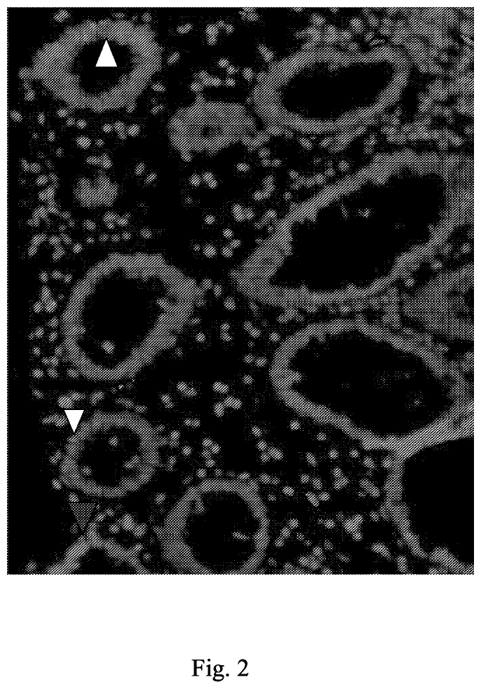

For the antibodies used in the steps described above, emission spectra for the dyes were as follows: DAPI was imaged at 455 nm, anti-CD3 was imaged at 555 nm, and anti-Caspase 1 was imaged at 488 nm. FIG. 2 shows a representative image of intestinal epithelial cells stained (i.e., immunostained) for activated caspase 1. The T cells present in the slide were identified by co-staining with an antibody that specifically bound to CD3, a cell surface molecule associated with the T cell receptor in T cells. In FIG. 2, the white arrow points to a green-stained intestinal epithelial cell that stained positive for expression of caspase 1, and the white arrow head (i.e., triangle) points to a red-stained T cell (an intra-epithelial lymphocyte, or IEL) that stained positive for expression of both CD3 and caspase 1.

### Example 3

### TUNEL Staining Protocol for Paraffin-Embedded Mucosal Biopsy Samples Using Commercially-Available Staining Kits

TUNEL (Terminal deoxynucleotidyl transferase dUTP nick end labeling) staining is a method for detecting DNA fragmentation by labeling the terminal ends of nucleic acids. Since apoptosis causes fragmentation of DNA, the TUNEL assay is a common method for DNA fragmentation that results from apoptotic signaling cascades. The assay relies on the presence of nicks in the DNA which can be identified by terminal deoxynucleotidyl transferase or TdT, an enzyme that will catalyze the addition of dUTPs that are secondarily labeled with a marker. Described below is a three step protocol for TUNEL staining of mucosal biopsy samples using commercially available staining kits.

### Step I. Deparaffinization

The slides containing mucosal biopsy samples, prepared as in Example 1, were placed in a rack, and the following sequential washes were performed in Coplin jars or another container:
Wash 1. Xylene: 2×5 minutes
Wash 2 and 3. 100% ethanol: 2×5 minutes
Wash 4. 95% ethanol: 3 minutes
Wash 5. 70% ethanol: 3 minutes
Wash 6. 50% ethanol: 3 minutes
Wash 7. Distilled H₂O: 2×3 minutes

The slides were kept in the distilled water until ready to perform antigen retrieval. The slides were not allowed to dry from this point onwards. Drying out may cause non-specific antibody binding and therefore high background staining on the tissue.

### Step II. Antigen Retrieval

1. A water bath and antigen retrieval solution (10 mM sodium citrate buffer) were preheated to 95° C. Sodium Citrate Buffer (10 mM sodium citrate, 0.05% Tween 20, pH 6.0) was made as follows: Tri-sodium citrate (dihydrate) 2.94 g and 1000 ml distilled water were mixed to dissolve the tri-sodium citrate. The pH was adjusted to 6.0 with 1N HCl. 0.5 ml Tween 20 was added to the solution, and the solution was mixed well and stored at 4° C.

2. The slides were placed in pre-heated antigen retrieval solution in a container (enough to cover the slides by a few centimeters). The use of glass containers was avoided as these may crack in the heat. A Tupperware or plastic container with a lid to prevent evaporation, or an empty box used to store pipette tips and having a lid, was used effectively. In certain embodiments, a weight was placed on the cover to prevent the container from floating around.

3. The slides were incubated for 20 minutes at 95° C.

4. When 20 minutes had elapsed, the container and slides were removed from the water bath. The slides were allowed to cool at room temperature while still immersed in the antigen retrieval solution before removing them from the container.

### Step II. Nuclear Staining

In one embodiment, the protocol provided by a commercially-available kit for staining was followed. For example, the abbreviated and adapted protocol for the Trevigen TACS® 2 TdT-Fluor In Situ Apoptosis Detection Kit, commercially available from Trevigen (Gaithersburg, Md.) Cat #: 4812-30-K was used.

1. The glass slides containing mucosal biopsy samples prepared as in Example 1 were immersed in 1× PBS for 10 minutes with gentle agitation.

2. The samples were covered with 50 μl of Proteinase K Solution for 15 minutes. The Proteinase K Solution (per sample) contained as follows: 50 μl Apoptosis Grade™ water and 1 μl Proteinase K.

3. The samples were washed in deionized water for 2 minutes. This wash was repeated a second time.

4. The samples were immersed in 1× TdT Labeling Buffer for 5 minutes. The TdT Labeling Buffer contained 45 ml Deionized Water and 5 ml 10× TdT Labeling Buffer (from the Trevigen kit).

5. The sample was covered with 50 μl of Labeling Reaction Mix (from the Trevigen kit) and incubated for 60 minutes at 37° C. in a humidity chamber. The Labeling Reaction Mix per sample contained 1 μl TdT dNTP, 1 μl 50× cation (Mg2+, Mn2+, or Co2+), 1 μl TdT Enzyme and 50 μl 1× TdT Labeling Buffer (from the Trevigen kit). Repeated cycles of freezing and thawing of the TdT enzyme were avoided.

6. The samples were immersed in 1× TdT Stop Buffer for 5 minutes. The TdT Stop Buffer contained 45 ml Deionized Water and 5 ml 10× TdT Stop Buffer (from the Trevigen kit)

7. The samples were washed twice in 1× PBS. Each wash was 2 minutes.

8. The samples were covered with 50 μl of Strep-Fluor Solution and incubated for 20 minutes in the dark. The Strep-Fluor Solution contained 200 μl 1× PBST (i.e., 1× PBS with 0.05% Tween 20) and 1 μl Strep-Fluorescein.

9. The samples were washed three times in 1× PBS. Each wash was 2 minutes.

10. Glass coverslips were mounted using 90% glycerol, and viewed under a fluorescence microscope using a 495 nm filter.

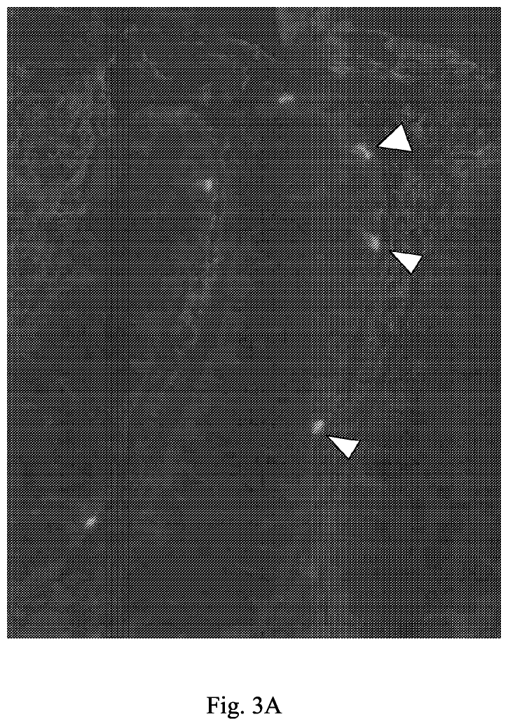

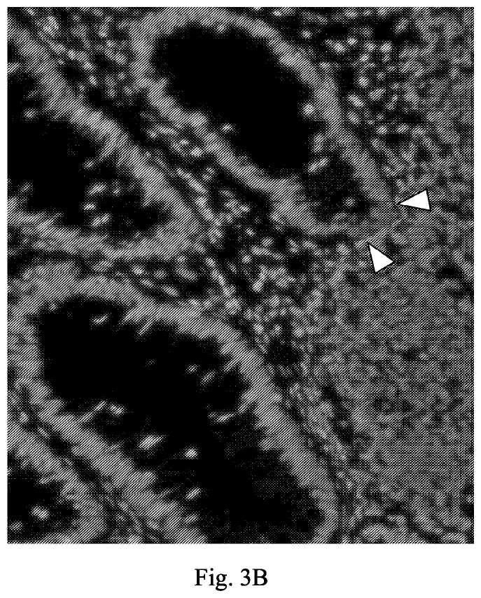

FIGS. 3A and 3B show representative images of intestinal epithelial cells stained (i.e., immunostained) for TUNEL (e.g., using the methods described in Example 3 above) (FIG. 3A) and activated caspase 3 (e.g., using the methods described in Example 2 above) (FIG. 3B). In FIG. 3A, the arrows point to intestinal epithelial cells staining positive for nuclear fragmentation using a commercial kit for TUNEL-positive cell staining. In FIG. 3B, the arrows point to intestinal epithelial cells staining positive for expression of caspase 3.

In some embodiments, staining with TUNEL stain (to detect dead or dying cells) was followed by staining for activated caspase-3, and the number of activated caspase-1 positive cells was determined by subtracting the number of caspase-3 positive cells from the total number of TUNEL positive cells.

In some embodiments, staining with TUNEL stain (to detect dead or dying cells) was followed by staining for activated caspase-1, and the number of activated caspase-3 positive cells was determined by subtracting the number of caspase-1 positive cells form the total number of TUNEL positive cells.

### Example 4

### Staining Using Quantum Dot Conjugated Antibody

Described below is a three step staining protocol using quantum dot conjugated antibody. Slides were prepared as described in Example 1.

### Step I. Deparaffinization

The slides were placed in a rack, and the following sequential washes were performed in Coplin jars or other containers:
Wash 1. Xylene: 2×5 minutes
Wash 2 and 3. 100% ethanol: 2×5 minutes
Wash 4. 95% ethanol: 3 minutes
Wash 5. 70% ethanol: 3 minutes
Wash 6. 50% ethanol: 3 minutes
Wash 7. Distilled H₂O: 2×3 minutes

The slides were kept in the distilled water until ready to perform antigen retrieval. The slides were not allowed to dry from this point onwards as drying out caused non-specific antibody binding and therefore high background staining on the tissue.

### Step II. Antigen Retrieval

1. A water bath and antigen retrieval solution (10 mM sodium citrate buffer) was pre-heated to 95° C.

Sodium Citrate Buffer (10 mM sodium citrate, 0.05% Tween 20, pH 6.0) was prepared by dissolving Tri-sodium citrate (dihydrate) (2.94 g) in 1000 ml distilled water. The solution was mixed to dissolve the tri-sodium citrate, and the pH was adjusted to 6.0 with 1N HCl.

0.5 ml Tween 20 was added to the solution, the solution was mixed well, and stored at 4° C.

2. The slides were placed in pre-heated antigen retrieval solution in a container (enough to cover the slides by a few centimeters). It is preferable to avoid using glass containers as these may crack in the heat. A plastic container or tupperware container with a lid was used to prevent evaporation. Alternatively, an empty box with a lid used for storing micropipette tips was used. In certain embodiments, a weight was placed on the cover of the container to prevent the container from moving/floating around in the antigen retrieval solution.

3. The slides were incubated for 20 minutes at 95° C.

4. When 20 minutes had elapsed, the slides and container were removed from the water bath. The slides were allowed to cool at room temperature, still immersed in the antigen retrieval solution, before being removed from the container.

5. The final step was to continue with the immunohistochemical staining protocol.

### Step III. Immunostaining

All incubations were carried out in a humidified chamber to avoid drying of the tissue.

A shallow, plastic box with a sealed lid and wet tissue paper in the bottom was used. The slides were kept off the paper and laid flat so that the reagents did not drain off.

1. The slides were washed in 1× PBS with 0.025% Triton X-100 for 5 minutes with gentle agitation. The wash was repeated for a total of 2 washes.

2. The slides were removed from the wash buffer and excess liquid was removed from the slides using a Kimwipe to dry the slides. A PAP pen or other similar pen was used to draw a circle around the tissue to create a hydrophobic barrier around the sample.

3. Approximately 100 μL of blocking solution was pipetted onto the tissue, ensuring that the tissue section was completely covered, and the tissues were incubated at room temperature for 2 hours. The blocking solution was 1× PBS, 10% normal goat serum and 1% BSA.

4. A Kimwipe was used to blot off any excess blocking solution from the tissue and 100 μL of primary antibody solution was added to each slide. The slides were incubated overnight at 4° C., keeping them away from light. The primary antibody solution was 1× PBS with 1% BSA containing a Quantum dot-labeled caspase-1 p20 antibody at a dilution of 1:250.

5. Excess antibody solution was blotted off and the slides were washed in 1× PBS for 5 minutes, with gentle agitation. The wash was repeated for a total of two washes. The washes were performed in the dark.

6. Slides were incubated in 1× PBS containing 0.3 ug/mL (0.654 nM) DAPI for 10 minutes with gentle agitation in the dark. In one embodiment, 12 uL of 5 mg/mL DAPI stock in 200 mL PBS was used for a final single step wash and stain.

7. Excess liquid was drained off, a Kimwipe or other lint free tissue was used to wipe around the sections, and coverslips were mounted onto the tissue using ProLong Diamond Antifade Mountant (Molecular Probes, cat# P36970). The slides were allowed to cure overnight at room temperature in the dark before imaging.

### Example 5

### Conjugation of Monoclonal Antibodies with Quantum Dots

Antibodies useful for the invention were conjugated to semiconductor quantum dots as described below.

All reagents and necessary components were purchased commercially, for example, monoclonal antibody: Cleaved Caspase-1 (Asp297)(D57A2) Rabbit mAb, Cell Signaling Technologies, Catalog #4199, Concentration: 182 μg/mL; and quantum dots selected for conjugation: Qdot® 625, with an emission spectra between 605 nm and 612 nm, Molecular Probes, Catalog #S10452. Quantum dots with an emission spectra in the range of 525 nm to 800 nm are also available, and were used to conjugate the monoclonal antibody of interest in certain embodiments.

### Step A: Antibody Concentration and Buffer Exchange

Antibodies with a concentration of less than 2 mg/mL were concentrated prior to conjugation with the quantum dot. In addition, the commercial antibody buffer contains sodium azide, which was removed for proper conjugation to occur.

450 μL of dH₂O was added to a 1.5 mL disposable ultrafiltration centrifugal microfuge tube with an insert containing a polyethersulfone (PES) membrane used for the concentration, desalting, and buffer exchange of antibodies and other proteins in solution (“the small antibody concentrator tube”), and the tube was capped. The small antibody concentrator was centrifuged for 6 minutes at 5000×g. The cap and the membrane of the concentrator were facing towards the center of the rotor of the centrifuge so that proper washing of the membrane could occur. After the centrifugation, the flow through was discarded.

A sufficient volume containing 100-125 μg of the antibody to be conjugated, in this case, the Cleaved Caspase-1 (Asp297)(D57A2) Rabbit mAb, Cell Signaling Technologies, Catalog #4199, was loaded into the small antibody concentrator. For the Cleaved Caspase-1 antibody, a minimum of 690 μL was required.

If the antibody volume that is loaded into the concentrator was less than 500 μL, the antibody was diluted to 500 μL by adding antibody preparation buffer provided in the kit (Qdot® 625, Molecular Probes, Catalog #S10452).

The antibody was centrifuged for 6 minutes at 5000×g, ensuring that the membrane of the small antibody concentrator was facing towards the center of the rotor to allow for optimal collection of the antibody onto the membrane. The flow through was discarded following the centrifugation.

450μL of antibody preparation buffer was added to the small antibody concentrator and centrifuged for 6 minutes at 5000×g.

The concentrated antibody was collected from the top half of the small antibody concentrator and placed into the microfuge collection tube provided. Approximately 50 μL of antibody was collected. If this volume was greater than 50 μL, an additional centrifugation of 3 minutes at 5000×g was performed to further concentrate the antibody to a volume of 50 μL.

### Step B: Removal of Terminal Galactose Residues from the Fragment Crystallizable (FC) Region of the Antibody

10 μL of (3-galactosidase enzyme was added to the 50 μL antibody solution from Step A and the tube containing the mixture was tightly wrapped with parafilm. The sample was incubated at 37° C. for 4 hours.

### Step C: Addition of Azide Moiety to Modify the Carbohydrate Domain of the Antibody

Azide modification solution was prepared by adding the following components to the microfuge tube containing UDP-GalNAz provided in the Qdot® 625 kit (Molecular Probes).
75 μL of dH₂0;
10 μL of 20× Tris buffer, pH 7.0;
25 μL of buffer additive; and
80 μL of GalT enzyme

The azide modification solution was briefly vortexed, and the 50 μL of concentrated antibody was added. A brief centrifugation of the tube was performed to ensure that the solution was at the bottom of the microfuge tube. The tube was wrapped in parafilm and incubated at 30° C. overnight.

### Step D: Purification of Azide-Modified Antibody

1× Tris buffer (pH 7.0) was prepared by adding 500 μL of 20× Tris (pH 7.0) to 9.5 mL of dH₂0 in a 15 mL centrifuge tube and vortexing gently to mix.

1 mL of the 1× Tris buffer was placed into a large 15 mL disposable ultrafiltration centrifugal conical tube with an insert containing a polyethersulfone (PES) membrane used for the concentration, desalting, and buffer exchange of antibodies and other proteins in solution (“the large antibody concentrator tube”). The tube and insert were centrifuged for 10 minutes at 1200× g, ensuring that the membrane of the concentrator was facing towards the center of the rotor to allow for optimal washing of the membrane. The flow through was discarded following the centrifugation.

1.75 mL of 1× Tris buffer and 250 μL of the concentrated antibody from paragraph 00188 above was added to the large antibody concentrator tube. The antibody mixture was centrifuged for 6 minutes at 1200× g, ensuring that the membrane of the concentrator was facing towards the center of the rotor to allow optimal collection of the antibody onto the membrane. The flow through was discarded following the centrifugation.

1.8 mL of 1× Tris buffer was added to the large antibody concentrator and the mixture was centrifuged for 10 minutes at 1200×g. The flow through was discarded following the centrifugation.

Step D was repeated once.

### Step E: Antibody Concentration

1.8 mL of 1× Tris buffer was added to the large antibody concentrator tube and centrifuged for 10 minutes at 1400× g. The flow through was discarded following the centrifugation. Approximately 80-120 μL of liquid remained in the upper portion of the large antibody concentrator tube.

To collect the antibody, the antibody concentrator tube was inverted into a clean 15 mL conical collection tube and centrifuged for 3 minutes at 1000× g.

The antibody was transferred into a clean and sterile 1.5 mL microfuge tube. If the final volume of the collected antibody was less than 100 μL, the antibody was diluted to a final volume of 100 μL with 20× Tris buffer (pH 7.0).

### Step F: Conjugation of Quantum Dot to Modified Antibody

50 μL of the DIBO modified quantum dot nanocrystal provided in the Qdot® 625 kit (Molecular Probes) was added to the azide-modified collected concentrated antibody collected (see paragraph 00198 above) and vortexed gently. The mixture was briefly centrifuged and incubated at 25° C. overnight.

Following incubation the antibody-quantum dot conjugate was stored at 2-8° C., protected from light. The antibody was not frozen. For long-term storage, 0.02% w/v of sodium azide was added to the antibody solution.

### Example 6

### Correlation of Leaky Gut Syndrome Caused by Caspase-1 Activation and Colorectal Neoplasia

The correlation of leaky gut syndrome caused by caspase-1 activation and colorectal neoplasia was determined as described below.

Experiments were performed with primary monoclonal antibody directed to activated caspase-1 or activated caspase-3, the antibody being conjugated to a quantum dot having a particular wavelength. A 620-nm Q-dot conjugated to activated caspase-1 antibody was used.

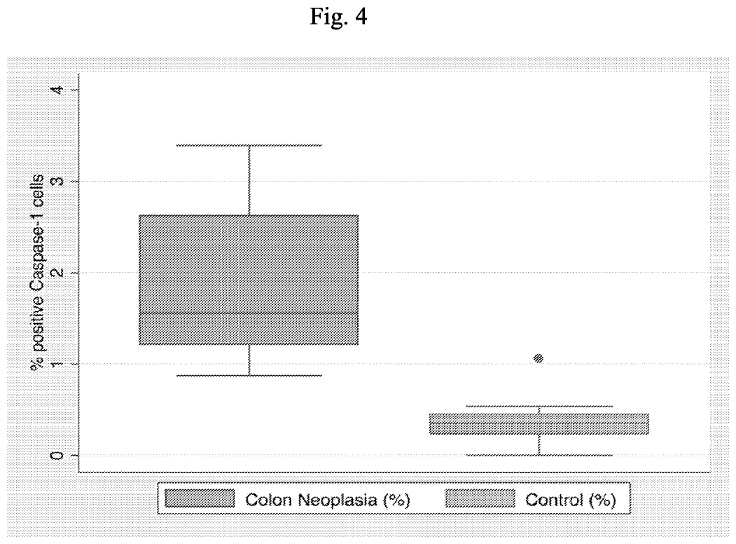

In a cohort of 16 patients undergoing screening colonoscopy, 10 patients had no lesions and served as healthy controls, and 6 patients exhibited colorectal neoplasia (colorectal neoplasia or adenomatous polyps). The median age for the 6 patients (3 males and 3 females) with colorectal neoplasia was 59 years. In this population of patients, the activated caspase-1 positive intestinal epithelial cells obtained by mucosal biopsy was 18.6 per 1000 epithelial cells counted, or 1.86% (FIG. 4). A population of 10 healthy control individuals, (3 males and 7 females) without any lesions found at the time of screening colonoscopy, had a median age of 54 years. The activated caspase-1 positive intestinal epithelial cells obtained by mucosal biopsy in this population was 5.9 per 1000 epithelial cells counted or 0.59%, which is significantly lower than those patients with colon cancer or adenomatous polyps (P<0.001) (FIG. 4). These data demonstrate that there is a correlation between leaky gut syndrome caused by caspase-1 activation and colorectal neoplasia.

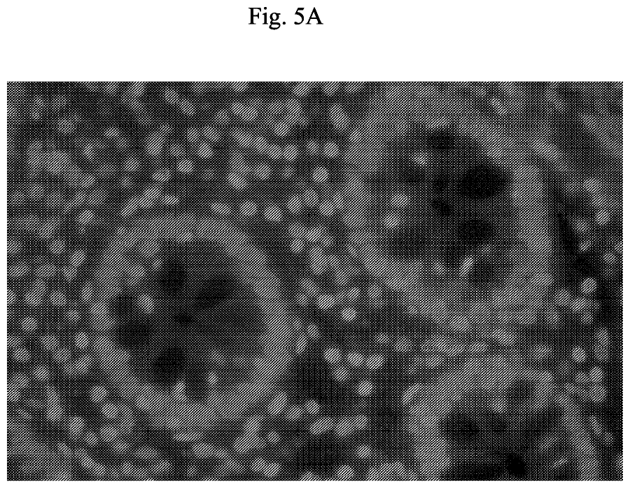

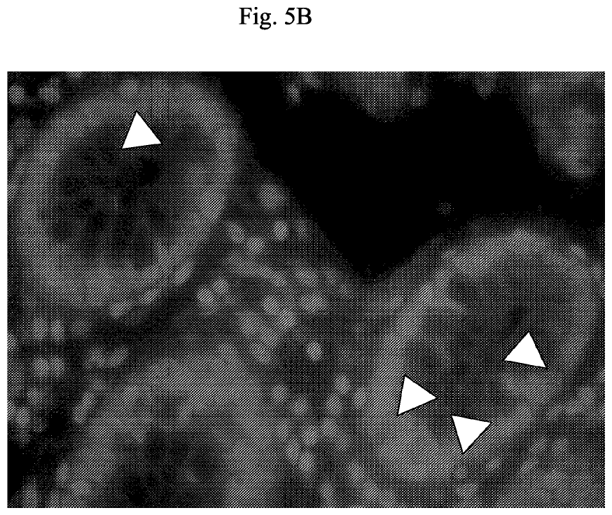

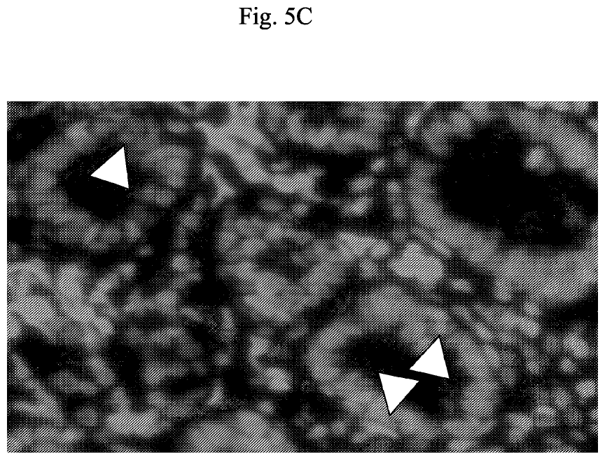

FIG. 5A presents representative images of intestinal biopsy samples from a healthy patient. FIGS. 5B and 5C are representative images of intestinal biopsy samples from a patient with colorectal neoplasia stained using primary monoclonal activated caspase-1 antibody (FIG. 5B); and stained using quantum dot conjugated antibody (FIG. C). White arrowheads indicate caspase-1 positive intestinal epithelial cells in the biopsy samples.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A method for detecting or identifying a leaky gut syndrome in a patient, comprising:
(a) providing a sample of a gastrointestinal (GI) barrier of the patient;
(b) analyzing the sample to determine the status of the GI barrier; and
(c) categorizing the patient GI barrier status as normal or abnormal, wherein an abnormal GI barrier status identifies the patient as having leaky gut syndrome.

2. The method of claim 1, wherein the patient has or is likely to develop a disease selected from the group consisting of a metabolic syndrome, cancer/neoplasia, an idiopathic inflammatory condition, a neurologic disorder, and a metabolic bone disease.

3. The method of claim 1, wherein the status of the GI barrier of the patient is analyzed by measuring an amount of activated caspase in intestinal epithelial cells of an intestinal barrier of the patient.

4. The method of claim 3, wherein activated caspase is measured by staining cells of the patient's GI barrier with a detectable marker conjugated to a caspase-1 specific antibody or with a probe comprising a detectable marker conjugated to a caspase-1 inhibitor.

5. The method of claim 3, wherein the activated caspase is activated caspase 1, activated caspase 3, or a combination of activated caspase 1 and activated caspase 3.

6. The method of claim 5, wherein an increase in the amount of activated caspase by about two to four fold in the patient as compared to the amount of activated caspase in intestinal epithelial cells of an intestinal barrier of one or more healthy subjects indicates that the patient GI barrier status is abnormal.

7. The method of claim 3, wherein the activated caspase is expressed as a ratio of an amount of expression of activated caspase 1 to an amount of expression of activated caspase 3.

8. The method of claim 7, wherein a ratio of activated caspase 1 to activated caspase 3 greater than 1.5 to 1 indicates that the patient GI barrier status is abnormal.

9. The method of claim 1, wherein the status of the GI barrier of the patient is analyzed by counting the number of gaps visualized by histological staining of an intestinal surface at the intestinal barrier.

10. The method of claim 9, wherein an increase in the number of gaps by about two to four fold in the patient as compared to the number of gaps in an intestinal surface at an intestinal barrier of one or more healthy subjects indicates that the patient GI barrier status is abnormal.

11. The method of claim 1, wherein the status of the GI barrier is analyzed using confocal laser endomicroscopy or multi-photo confocal microscopy of the GI barrier.

12. The method of claim 1, wherein the GI barrier is selected from the group consisting of a buccal mucosa barrier, an oropharyngeal barrier, and an intestinal barrier.

13. The method of claim 1, wherein the leaky gut syndrome is colorectal neoplasia

14. A method for detecting or identifying a leaky gut syndrome in a patient, comprising:
a. staining gastrointestinal (GI) cells of the patient with a detectable marker conjugated to a caspase-1 specific antibody;
b. examining the stained GI cells of the patient for the presence of elevated levels of bound detectable antibody relative to similarly stained GI cells from a healthy individual as evidence of above-normal levels of caspase-1 associated with the patient GI barrier cells, wherein elevated levels of caspase-1 identifies the patients as having leaky gut syndrome.

15. The method of claim 14, wherein the GI barrier is selected from the group consisting of a buccal mucosa barrier, an oropharyngeal barrier, and an intestinal barrier.

16. The method of claim 14, wherein said staining comprises the steps of (i) obtaining patient intestinal epithelial cells from the patient by biopsy or aspiration, and (ii) staining the cells in vitro.

17. The method of claim 14, further comprising staining the GI barrier cells with a detectable marker conjugated to a caspase-3 specific antibody.

18. The method of claim 17 wherein the detectable marker is fluorescent, and said examining is performed by fluorescence microscopy, multi-photon microscopy, confocal laser endomicroscopy, fluorescence flow cytometry or by using a fluorescence plate reader.

19. The method of claim 1 wherein said staining includes applying the detectable marker conjugated to the caspase-1 antibody to intestinal epithelial cells in the patient's intestine, and said examining includes visualizing the stained cells endoscopically.

20. The method of claim 1, wherein the detectable marker is a quantum dot.

21. The method of claim 20, wherein the quantum dot has an emission spectra of 625 nm, or in the range of 525 nm to 800 nm or 605 nm and 612 nm.

22. The method of claim 14, further comprising the step of identifying dead or dying cells

23. The method of claim 22, wherein dead or dying cells are identified using the TUNEL assay.

24. The method of claim 1, wherein an increase in the amount of caspase-1 by about two to four fold in the patient as compared to the amount of caspase-1 in GI barrier cells of a GI barrier of one or more healthy subjects indicates that the patient has a leaky gut syndrome.

25. The method of claim 1, wherein the patient is human

26. The method of claim 14, wherein the leaky gut syndrome is colorectal neoplasia.

27. The method of claim 14, wherein the patient has or is likely to develop a disease selected from the group consisting of a metabolic syndrome, cancer/neoplasia, an idiopathic inflammatory condition, a neurologic disorder, and a metabolic bone disease.

28. A method for detecting or identifying a leaky gut syndrome in a patient, comprising:
a. staining gastrointestinal (GI) cells of the patient with a detectable marker conjugated to a caspase-1 inhibitor;
b. examining the stained GI cells of the patient for the presence of elevated levels of bound detectable antibody relative to similarly stained GI cells from a healthy individual as evidence of above-normal levels of caspase-1 associated with the patient GI barrier cells, wherein elevated levels of caspase-1 identifies the patients as having leaky gut syndrome.


SEQUENCE LISTING (2 sequences)

SEQ ID NO: 1 - protein, 4 aa, artificial
  synthetic
YVAD

SEQ ID NO: 2 - protein, 8 aa, artificial
  synthetic
GGGGYVAD